(19) United States
(12) Patent Application Publication
(10) Pub. No.: US 2026/0137786 A1
(43) Pub. Date: May 21, 2026

(54) FLUORESCENT COMPOUNDS FOR SYNERGISTIC PHOTODYNAMIC AND PHOTOTHERMAL CANCER THERANOSTIC

(71) Applicant: The Hong Kong University of Science and Technology, Hong Kong (CN)

(72) Inventors: BEN-ZHONG TANG, Hong Kong (CN); HUILIN XIE, Hong Kong (CN); TSZ KIN KWOK, Hong Kong (CN)

(21) Appl. No.: 19/121,244

(22) PCT Filed: Sep. 28, 2023
(86) PCT No.: PCT/CN2023/122882

§ 371 Date: Apr. 15, 2025

Related U.S. Application Data
(60) Provisional application No. 63/416,936, filed on Oct. 17, 2022.

(51) Int. Cl. A61K 41/00; A61K 9/00; A61K 9/51; A61K 49/00; A61P 35/00; C07D 513/04

(52) U.S. Cl. CPC A61K 41/008; A61K 9/0019; A61K 9/5146; A61K 49/0021; A61P 35/00; C07D 513/04

(57) ABSTRACT

The present invention discloses aggregation-induced emission fluorescent compounds with near-infrared (NIR) emission. The present invention also shows application for imaging-guided phototheranostic with the integration of both photodynamic therapies and photothermal therapies. The in vitro and in vivo verification further confirmed that this system exhibits outstanding fluorescence-guided phototheranostics ability to eliminate tumors effectively.

### CROSS REFERENCE TO RELATED APPLICATIONS

This application claims priority to and the benefit of U.S. Provisional Patent Application Ser. No. 63/416,936 filed on Oct. 17, 2022, the disclosure of which is incorporated by reference in its entirety.

### TECHNICAL FIELD

The present invention relates to aggregated-induced emission fluorescent compounds with near-infrared (NIR) emission and applies them into imaging-guided phototheranostic.

### BACKGROUND

Cancer, as one of the most severe health issues in the world, has caused an extremely high mortality rate. Although multifarious interventions, including chemotherapy, radiotherapy, and surgery, have been used to treat cancer, their severe side effects and grim therapeutics outcomes are still far from satisfactory. Therefore, developing more effective cancer therapies is essential but challenging. Fluorescence-guided phototheranostic, which utilizes photoirradiation to achieve cancer diagnostics and therapeutics coinstantaneously, has emerged as a promising tool because of its controllable, non-invasive, and spatio-temporal therapeutic process. However, robust phototherapy systems, which can simultaneously be equipped with diagnostic and therapeutic abilities, are extremely scarce and highly demanded due to the difficulty of tackling the complicated energy decay pathways upon excitation.

It is well known that in a photoexcitation process, one typical radiative pathway for the molecule to deal with the energy received after excitation is fluorescence (FL). Another pathway is non-radiative dissipation to afford the photothermal effect, which could be used as photothermal therapy (PTT). Besides, some photosensitizers could achieve photodynamic therapy (PDT) resulting from the production of reactive oxygen species (ROS) from the intersystem crossing (ISC).

In phototheranostic, PTT and PDT are two promising therapies with negligible drug resistance, noninvasiveness, and high sensitivity. However, using PDT or PTT alone is not always sufficient for tumor elimination because the hypoxic microenvironment of tumor tissue may limit the PDT efficiency and the temperature increase is not enough to obliterate the lesion. Accordingly, advanced materials to integrate PDT and PTT cooperatively is highly desirable for cancer therapy.

### SUMMARY

The present invention provided aggregation-induced emission fluorescent compounds with near-infrared (NIR) emission. The present invention also shows application for imaging-guided phototheranostic with the integration of both photodynamic therapies and photothermal therapies. The in vitro and in vivo verification further confirmed that this system exhibits outstanding fluorescence-guided phototheranostics ability to eliminate tumors effectively.

In one embodiment, a fluorescent compound is provided. The fluorescent compound exhibits aggregation-induced emission properties, the fluorescent compound having a backbone structural formula selected from the group consisting of:

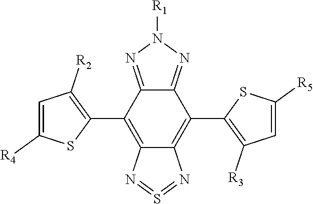

wherein each R₁, R₂, and R₃ is independently selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C(O)—O—, —CR5=CR6-, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each R₄ and R₅ is independently selected from the group consisting of:

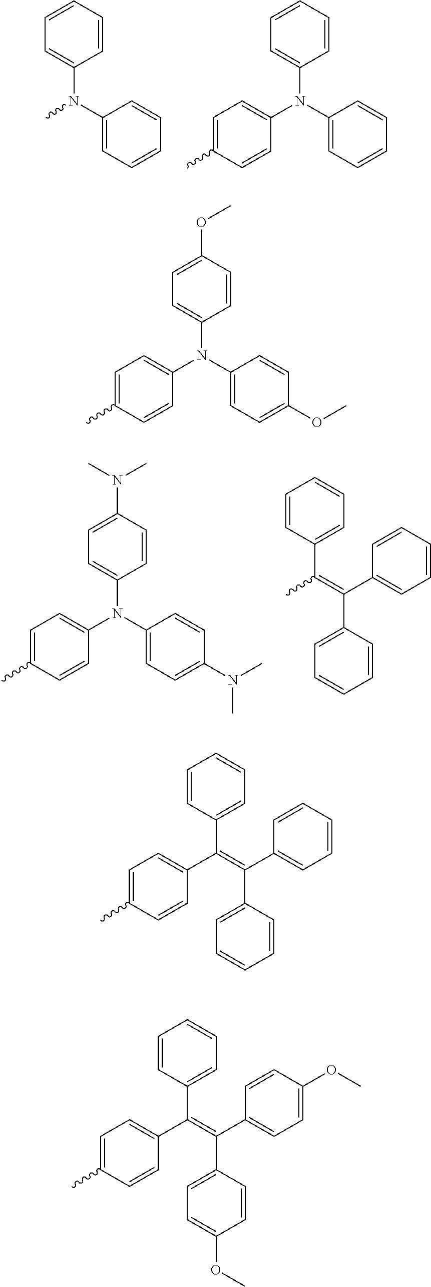

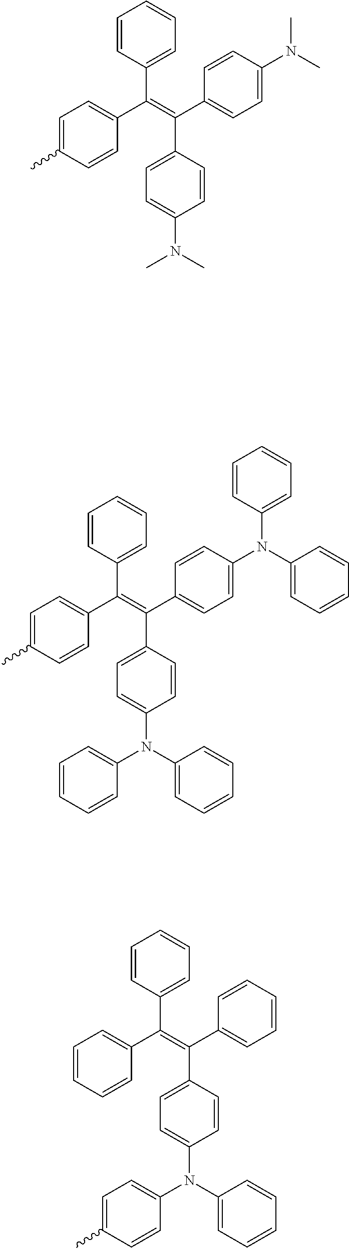

In another embodiment, a theranostic agent is provided. The theranostic agent comprising the above-mentioned fluorescent compound in nanoparticle form and a polymer matrix encapsulating the fluorescent compound.

In still another embodiment, a method of killing cancer cells is provided. The method comprising:
contacting a target cancer cell with the above-mentioned theranostic agent;
imaging the target cancer cell while the theranostic agent contacts the target cancer cell using an imaging method selected from the group consisting of fluorescence microscopy, bioluminescence imaging, and confocal laser scanning microscopy; and
subjecting the target cancer cell to light irradiation while the theranostic agent is contacting the target cancer cell to kill the target cancer cell.

In further another embodiment, a method of stopping or inhibiting tumor growth in a mammal is provided. The method comprising:
administering the above-mentioned theranostic agent to the mammal;
contacting a tumor site with the theranostic agent;
locating the tumor site using an imaging method after the tumor site is contacted with the theranostic agent; and
subjecting the tumor site to light irradiation while the theranostic agent is present at the tumor site to stop or inhibit the growth of the tumor.

The above description is only an outline of the technical schemes of the present invention. Preferred embodiments of the present invention are provided below in conjunction with the attached drawings to enable one with ordinary skill in the art to better understand said and other objectives, features and advantages of the present invention and to make the present invention accordingly.

### BRIEF DESCRIPTION OF THE DRAWINGS

The present invention can be more fully understood by reading the following detailed description of the preferred embodiments, with reference made to the accompanying drawings, wherein:

FIG. 1 Synthetic route of TPA-TBT and TPE-TBT.

FIG. 2 Structure of TPA-TBT and TPE-TBT.

FIG. 3a Normalized absorption and FIG. 3b Normalized PL spectra of the TPA-TBT and TPE-TBT (10 μM) in THE solution. FIG. 3c Plots of relative PL intensity (I/I₀) of TPA-TBT at 830 nm and TPE-TBT at 750 nm in different water fractions. FIG. 3d Quantum yield of the compounds in THE solution, nanoparticles, and solid state. FIG. 3e ROS generation capability of TPA-TBT and TPE-TBT NPs under white light irradiation. FIG. 3f Photothermal performance of the NPs under the irradiation of 660 nm laser (1 W cm⁻²).

FIG. 4 DLS spectrum of TPA-TBT nanoparticles.

FIG. 5 DLS spectrum of TPE-TBT nanoparticles.

Relative viability of 293T, Hela, and 4T1 cells after incubation with TPA-TBT NPs in different concentrations for FIG. 6a 0 h and FIG. 6b 24 h, then irradiated with white light (11 mW cm⁻²) for 10 min. FIG. 6c Flow cytometry analysis of 4T1 cells after various treatments, including cells without treatment (control), TPA-TBT NPs-treated cells, 660 nm laser-treated, and cells treated with NPs and laser. Laser power: 1 W cm⁻².

FIG. 7 CLSM imaging of intracellular ROS by H₂DCF-DA staining for 4T1 cells under different experimental conditions, including control, TPA-TBT NPs, TPA-TBT NPs+White light (White light, 11 mW cm⁻², 10 min; Excitation: 488 nm; Filter: 500-550 nm.), DAPI is a nuclear dye (Excitation: 405 nm; Filter: 460-500 nm).

FIG. 8a Time-dependent NIR FLI of 4T1 tumor-bearing mice after injection of TPA-TBT NPs. FIG. 8b Quantitative fluorescence intensity of tumor tissues at different monitoring times after administration of the NPs. FIG. 8c Infrared thermal images of 4T1 tumor-bearing mice under 660 nm laser irradiation (0.5 W cm⁻²) for different times after injection of the NPs. FIG. 8d temperature changes of 4T1 tumor-bearing mice after intravenous injection of TPA-TBT NPs, and PBS under 660 nm laser irradiation (0.5 W cm⁻²) for 10 min. FIG. 8e The relative tumor volume changes after various treatments. Inset: representative tumor images of the four groups after 14 days of treatment (left to right: PBS, TPA-TBT NPs, laser, TPA-TBT NPs+laser). FIG. 8f Photos of tumor-bearing mice from different groups. FIG. 8g H&E and TUNEL staining analyses of tumor tissues after various treatments.

FIG. 9 The representative NIR-I fluorescence images of ex vivo organs collected from 4T1 tumor-bearing mice injected with TPA-TBT NPs at 12 h and the quantitative analysis of mean fluorescence intensity.

FIG. 10. In vivo photothermal images of 4T1 tumor-bearing mice after administration of PBS or TPA-TBT NPs (1.0 mg mL⁻¹, 200 μL) under 660 nm NIR laser irradiation.

FIG. 11. The tumor images of the four groups after 14 days of treatment (left to right: PBS, TPA-TBT NPs, laser, TPA-TBT NPs+laser).

FIG. 12 Body weight of mice in 14 days after different treatments. No obvious side effect was observed in all groups.

FIG. 13 Blood routine data of BALB/c female mice after 7 or 14 days treatment with PBS or TPA-TBT NPs, including white blood cell (WBC), red blood cell (RBC), platelet (PLT), lymphocyte (Lymph), hemoglobin (HGB), procalcitonin (PCT). (n=3 per group)

FIG. 14 Blood biochemical data of BALB/c female mice after 7 or 14 days treatment with PBS or TPA-TBT NPs, including alanine amiotransferase (ALT), aspartate aminotransferase (AST), total protein (TP), albumin (ALB), creatinine (CR), total cholesterol (TCH) and urea (UREA). (n=3 per group)

FIG. 15 H&E stained images of major organs including the heart, liver, spleen, lung and kidney of BALB/c female mice after 7 or 14 days treatment with PBS or TPA-TBT NPs. Scale bar: 100 μm.

### DETAILED DESCRIPTION

### Definitions

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

The term “λₑₓ” as used herein refers to excitation wavelength.

The phrase “aggregation caused quenching” or “ACQ” as used herein refers to the phenomenon wherein the aggregation of π-conjugated fluorophores significantly decreases the fluorescence intensity of the fluorophores. The aggregate formation is said to “quench” light emission of the fluorophores.

The phrase “aggregation induced emission” or “AIE” as used herein refers to the phenomenon manifested by compounds exhibiting significant enhancement of light-emission upon aggregation in the amorphous or crystalline (solid) states whereas they exhibit weak or almost no emission in dilute solutions.

“Emission intensity” as used herein refers to the magnitude of fluorescence/phosphorescence normally obtained from a fluorescence spectrometer or fluorescence microscopy measurement; “fluorophore” or “fluorogen” as used herein refers to a molecule which exhibits fluorescence; “luminogen” or “luminophore” as used herein refers to a molecule which exhibits luminescence; and “AIEgen” as used herein refers to a molecule exhibiting AIE characteristics.

As used herein, a “donor” material refers to an organic material, for example, an organic nanoparticle material, having holes as the majority current or charge carriers.

As used herein, an “acceptor” material refers to an organic material, for example, an organic nanoparticle material, having electrons as the majority current or charge carriers.

As used herein, a “theranostic agent” refers to an organic material, for example, an organic nanoparticle material, having both diagnostic and therapeutic capabilities.

Unless defined otherwise, all technical and scientific terms used herein have the same meaning as commonly understood to one of ordinary skill in the art to which the presently described subject matter pertains.

### Fluorescent Compounds and Compositions

Among various phototheranostic agents, fluorescent nanoparticles (NPs) are particularly desirable due to their associated high sensitivity and temporal resolution of fluorescence imaging, on-demand and in situ signature of photodynamic therapy (PDT), as well as unique enhanced permeability and retention (EPR) effects. To meet the requirements of ideal cancer phototheranostics, the fluorescent NPs must have several qualities, including sufficiently high near-infrared (NIR) emission (>650 nm); reactive oxygen species (ROS) generation efficiency of the fluorescent component within the NPs; strong resistance to photobleaching; negligible cytotoxicity and in vivo toxicity; and suitable NP size and surface chemistry, permitting prominent EPR effect.

Aggregation-induced emission luminogens (AIEgens) have recently emerged as an alternative fluorescent material to construct fluorescent NPs, which perfectly address the challenge of ACQ and exhibit low in vivo side toxicities. AIEgens are often non-emissive in solution due to the consumption of the excited state energy via non-radiative relaxation by intramolecular motion. Upon aggregation, such relaxation from the lowest excited singlet state (S₁) to the ground state (S₀) is largely restricted due to steric hindrance, leading to the energy of S₁ going through the fluorescence pathway to S₀. This uncommon feature makes AIEgens ideal for fabrication of fluorescent NPs with ultrahigh brightness and photobleaching threshold.

The present subject matter contemplates a fluorescent compound having aggregation-induced emission (AIE) characteristics and exhibiting near-infrared emission. Near-infrared (NIR) fluorescence imaging exhibits lower tissue autofluorescence interference and deeper tissue penetration depth than visible light, so it has drawn extensive attention. The compound can be in nanoparticle form.

Also provided are compositions including the fluorescent compound and polymer matrix encapsulating the fluorescent compound. The compositions can be in nanoparticle form. Compositions including the nanoparticle form of the fluorescent compound and the polymer matrix are also referred to herein as “theranostic agents”.

Encapsulating the fluorescent compound in a polymer matrix can enhance the intra-particle microenvironment and thereby provide enhanced fluorescence and ROS generation capacity in vivo.

In an embodiment, the compounds have a backbone structural formula selected from the group consisting of:

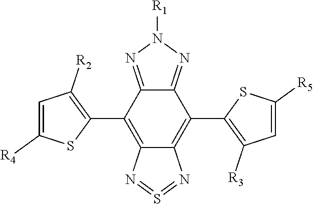

wherein each R₁, R₂, and R₃ is independently selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C (O)—O—, —CR5=CR6-, or —C—C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each R₄ and R₅ is independently selected from the group consisting of:

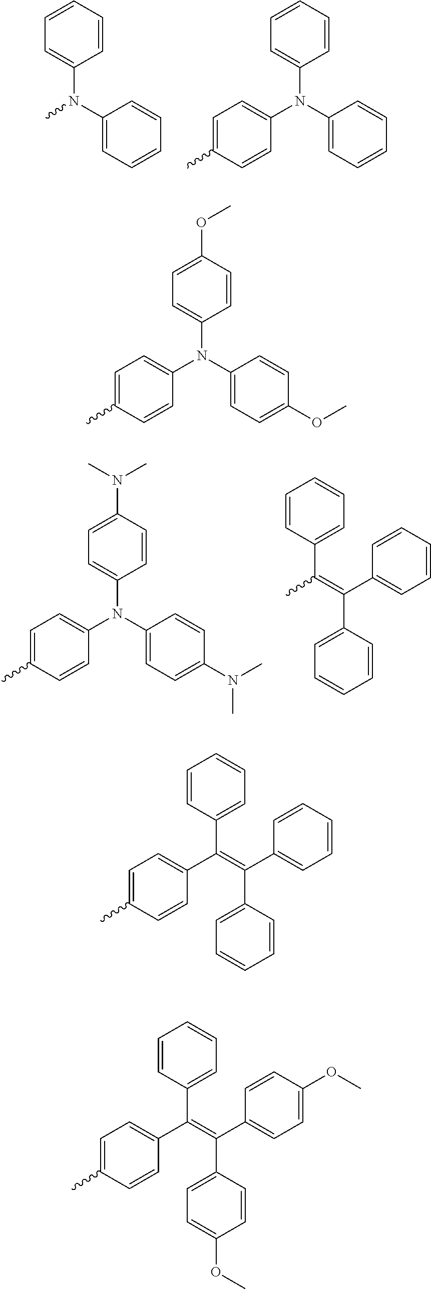

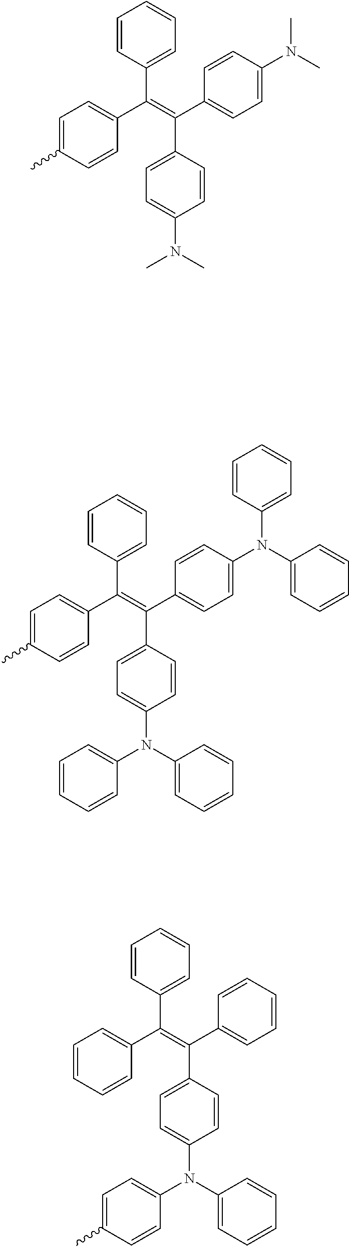

Additionally, R₁, R₂, and R₃ can be branched alkyl with 2-40 C atoms.

In a further embodiment, the fluorescent compound the fluorescent compound is selected from the group consisting of:

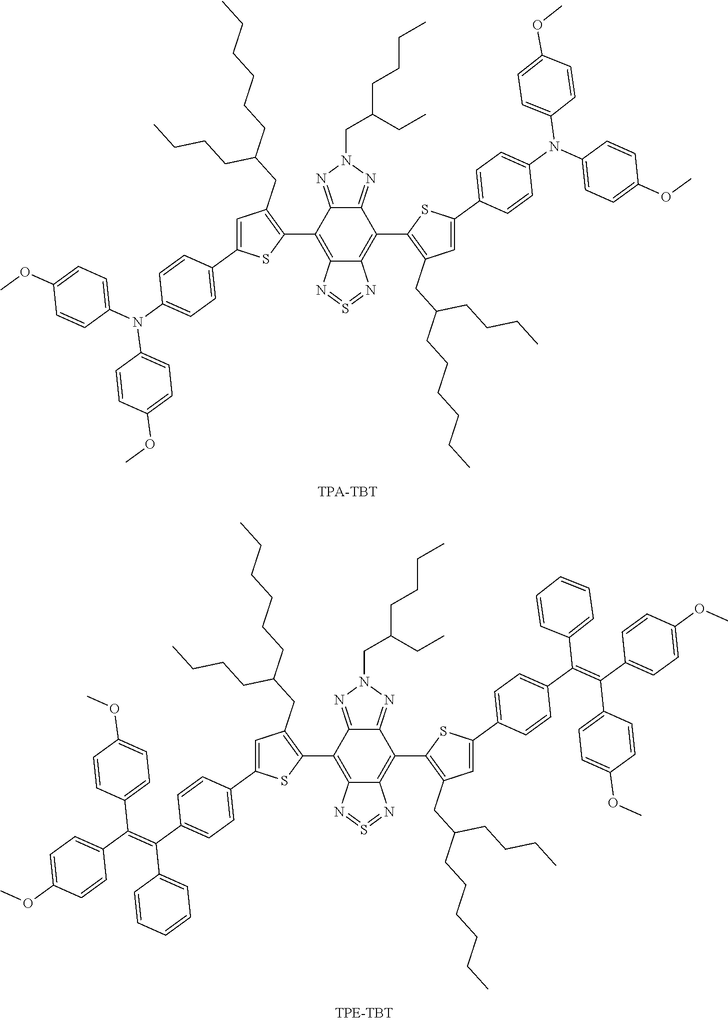

In yet another embodiment, the present subject matter relates to a fluorescent nanoparticle composition, comprising a fluorescent compound exhibiting aggregation induced emission properties and a polymer matrix, the polymer matrix encapsulating the fluorescent compound, the fluorescent compound having a backbone structural formula selected from the group consisting of:

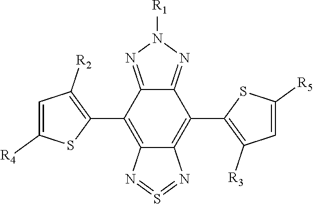

wherein each R₁, R₂, and R₃ is independently selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C (O)—O—, —CR5=CR6-, or —C—C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each R₄ and R₅ is independently selected from the group consisting of:

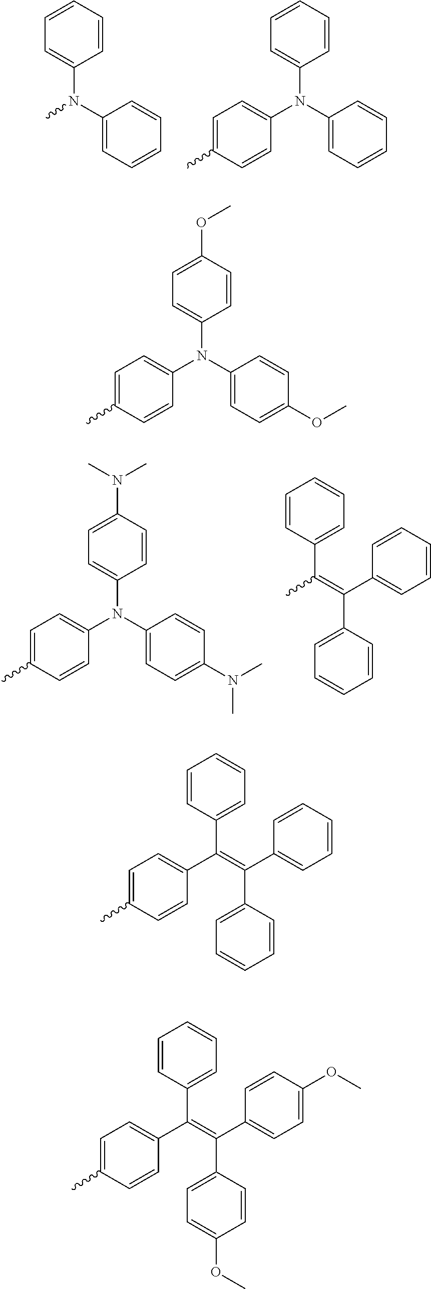

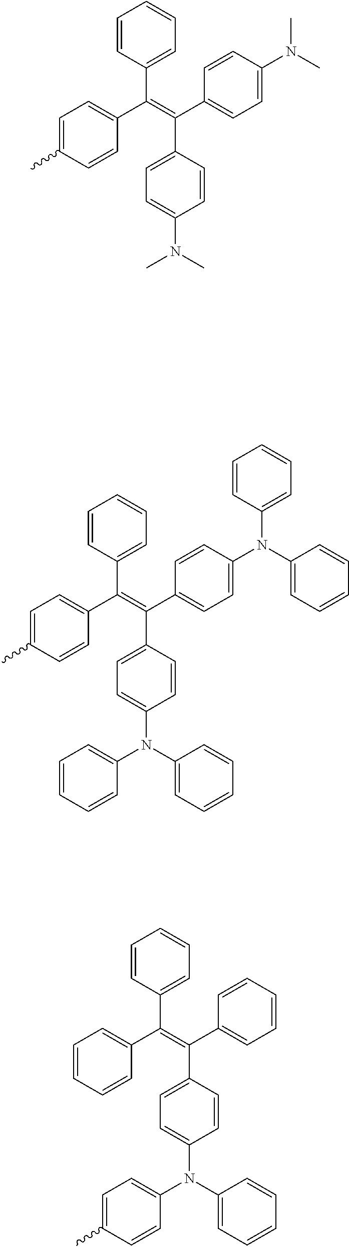

Additionally, R₁, R₂, and R₃ can be branched alkyl with 2-40 C atoms.

In a further embodiment, the fluorescent compound of the composition is selected from the group consisting of:

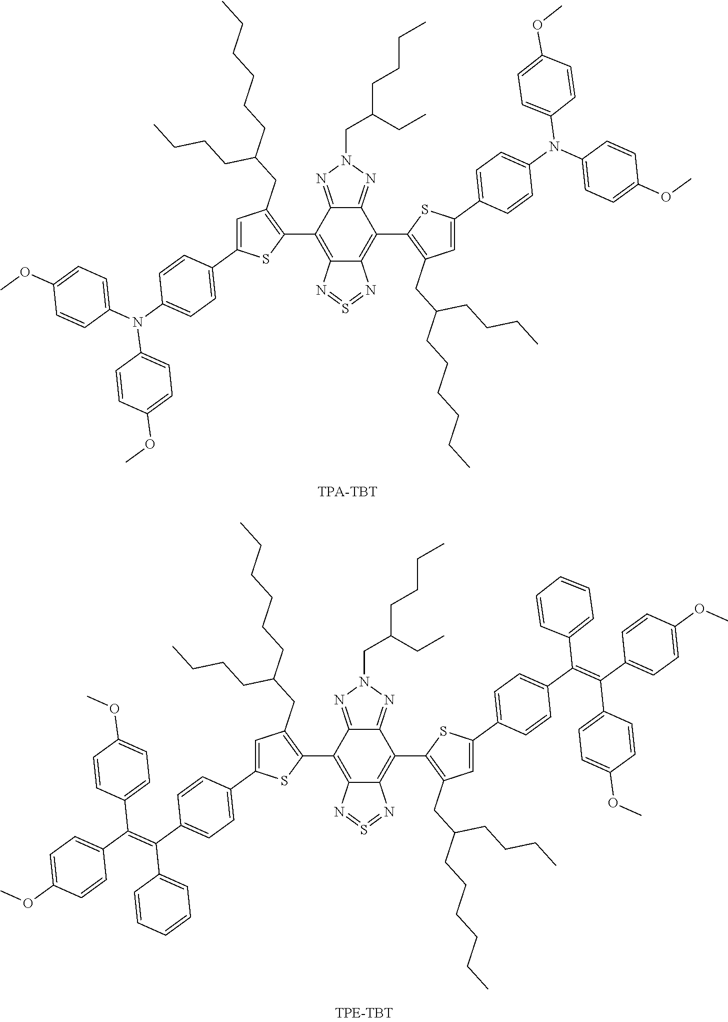

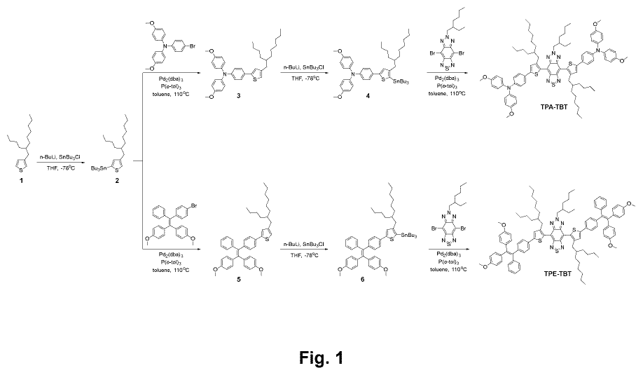

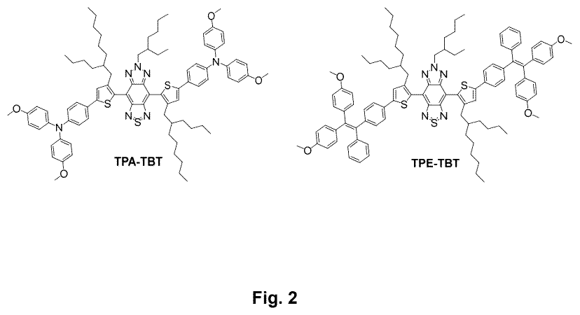

An exemplary reaction scheme for preparing TPA-TBT and TPE-TBT compounds is illustrated in FIG. 1.

As mentioned previously, in a photoexcitation process, one typical radiative pathway for the molecule to deal with the energy received after excitation is fluorescence (FL). Another pathway is non-radiative dissipation to afford the photothermal effect, which could be used as photothermal therapy (PTT). Besides, some photosensitizers could achieve photodynamic treatment (PDT) resulting from the production of reactive oxygen species (ROS) from the intersystem crossing (ISC). Due to the total energy being stationary, there should be a balance that could eclectically make use of all the energy dissipation processes to realize the most efficient imaging-guided phototherapy by using one single molecule.

In a preferred embodiment of this invention, we provided a one-for-all AIE system for imaging-guided phototheranostic with the integration of both PDT and PTT. By rational comparing the donor moieties, the strong electron donation group triphenylamine (TPA) was chosen, along with the thiadiazolobenzotriazole (TBT), to achieve a high quantum yield-NIR emissive AIEgen TPA-TBT. The TPA-TBT nanoparticles (TPA-TBT NPs) were fabricated for excellent biocompatibility, AIE effect, and enrichment in solid tumors. And we found that TPA-TBT NPs contributed to a long emission wavelength, high fluorescence quantum yield, excellent photothermal conversion efficiency, and remarkable ROS generation ability.

Cancer Diagnostics and/or Cancer Therapy

The theranostic agents described herein can be beneficial in cancer diagnostic and phototheranostic applications, particularly with respect to NIR imaging-guided cancer surgery with the integration of both PDT and PTT. Image-guided cancer surgery using NIR fluorescence has been verified to be feasible during clinical cancer surgery, and holds great promise for successful outcomes in cancer surgery. The theranostic agents described herein can be used as efficient NIR fluorescent probes that meet the necessary requirements of image-guided cancer surgery.

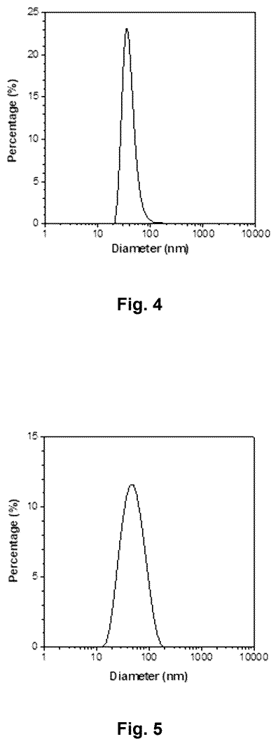

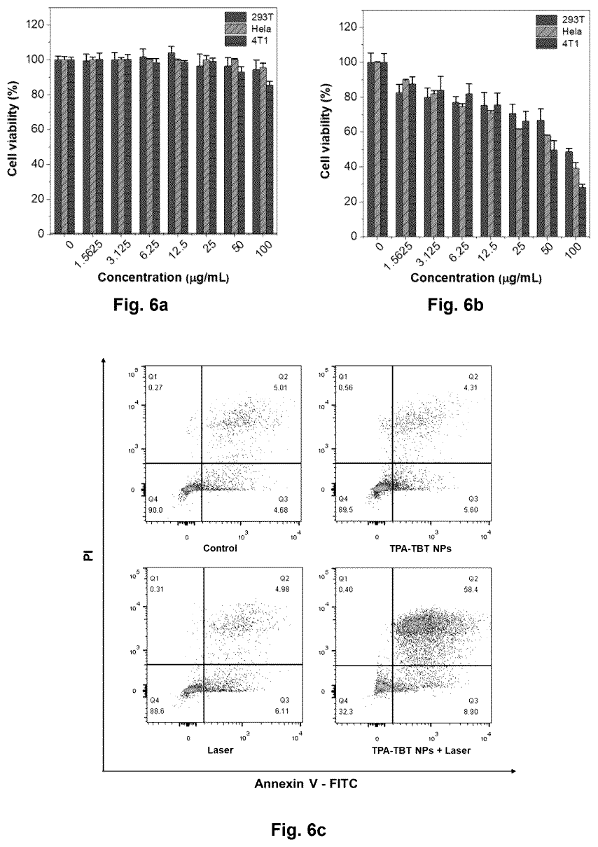

As described herein, TPA-TBT and TPE-TBT were fabricated into nanoparticles (NPs) through the nanoprecipitation method using DSPE-PEG2000 as an encapsulation matrix. The hydrodynamic diameters of the two NPs were confirmed by dynamic light scattering (DLS). The result shows the TPA-TBT and TPE-TBT NPs are well dispersed in water with diameters of around 37 nm and 45 nm, respectively (FIG. 4 and FIG. 5).

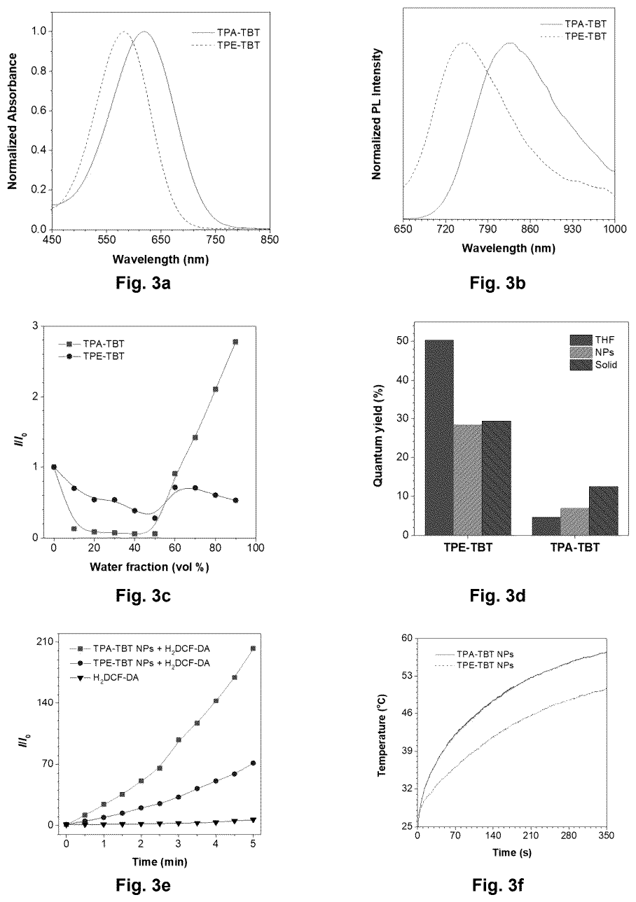

The fluorescence quantum yield (QY) of the two compounds in the solid state, NPs, THF, and other solvents, were also measured (FIG. 3d). The result shows that the TPA-TBT NPs have a high absolute QY of 7%, while TPE-TBT NPs have an even higher QY of 28%. The QY of TPA-TBT in the solid state is 13%, which is higher than that in NPs and THE solution (5%), while TPE-TBT has remarkable QY in the solution state, indicating TPE-TBT shows a more significant TICT effect and active radioactive decay.

ROS production efficiency and PCE were investigated to further explore the phototherapy potential of these AIEgens. We utilized a common ROS indicator Dichlorofluorescin Diace (H₂DCF-DA), whose fluorescence can be sensitively activated by ROS, to indicate the overall ROS generation. From FIG. 3e, the fluorescence intensity of H₂DCF-DA intensified rapidly in the presence of TPA-TBT NPs. Finally, it reached around 210-fold more robust than the initial intensity after 5 min upon white light irradiation, displaying a much higher ROS production efficiency than TPE-TBT NPs.

In addition, TPA-TBT NPs exhibit better photothermal performance. As shown in FIG. 3f, the temperature of TPA-TBT NPs reached around 57° C. upon the irradiation of 660 nm laser in the 350s, with a PCE of 44%. While the PCE of TPE-TBT NPs is only 30%. Considering all the aspects, TPA-TBT NPs show longer fluorescence emission wavelength, higher ROS generation efficiency, and better photothermal efficiency. Therefore, TPA-TBT NPs are more promising for applications in the field of phototheranostic for synergistic cancer therapy.

According to an embodiment, the present subject matter relates to a method of killing cancer cells, which can include contacting the theranostic agent with a target cancer cell, imaging the target cancer cell while the theranostic agent contacts the target cancer cell, and subjecting the target cancer cell to light irradiation while the theranostic agent contacts the target cancer cell to kill the target cancer cell. The imaging method can be selected from fluorescence microscopy, bioluminescence imaging, and confocal laser scanning microscopy. The theranostic agent can be combined with a buffer solution prior to contacting the target cancer cell. The target cancer cell can be in live mammals.

In the step of subjecting the target cancer cell to light irradiation while the theranostic agent is contacting the target cancer cell absorbs light and generates reactive oxygen species to kill the target cancer cell.

In the step of subjecting the target cancer cell to light irradiation while the theranostic agent is contacting the target cancer cell converts light to heat to kill the target cancer cell.

The light irradiation can be white light irradiation, red light irradiation or laser irradiation.

According to an embodiment, the present subject matter relates to a method of stopping or inhibiting tumor growth in a mammal, which can include administering the theranostic agent to the mammal; contacting a tumor site with the theranostic agent; locating the tumor site using an imaging method after the tumor site is contacted with the theranostic agent; and subjecting the tumor site to light irradiation while the theranostic agent is present at the tumor site to stop or inhibit the growth of the tumor. The theranostic agent can be administered by injection. The imaging method comprises at least one of fluorescence microscopy, bioluminescence imaging, and confocal laser scanning microscopy.

In the step of subjecting the tumor site to light irradiation while the theranostic agent is present at the tumor site absorbs light and generates reactive oxygen species to stop or inhibit the growth of the tumor.

In the step of subjecting the tumor site to light irradiation while the theranostic agent is present at the tumor site converts light to heat to stop or inhibit the growth of the tumor.

The light irradiation can be white light irradiation, red light irradiation or laser irradiation.

The following examples are provided to illustrate further and to facilitate the understanding of the present teachings and are not in any way intended to limit the invention.

### EXAMPLES

### Example 1—Synthesis and Characterization

The Synthetic route of TPA-TBT and TPE-TBT are shown in FIG. 1.

### Synthesis of tributyl(4-(2-butyloctyl)thiophen-2-yl)stannane (Compound 2)

To a solution of 3-(2-butyloctyl)thiophene (compound 1, 2.00 g, 7.93 mmol) in anhydrous THE (80 mL) was added n-BuLi (3.8 mL, 2.5 M in hexane) at −78° C. under N₂ atmosphere. The mixture was stirred at the same temperature for one hour, followed by the addition of tributyltin chloride (3.35 g, 10.31 mmol). The reaction was allowed to slowly warm up to room temperature and stirred overnight. The reaction mixture was quenched by aqueous KF solution, washed with water and brine, and dried over Na₂SO₄. The crude product was obtained by concentration under reduced pressure and used without further purification.

### Synthesis of 4-(4-(2-butyloctyl)thiophen-2-yl)-N,N-bis(4-methoxyphenyl)aniline (Compound 3)

A mixture of 4-bromo-N,N-bis(4-methoxyphenyl)aniline (1.00 g, 2.61 mmol), compound 2 (1.70 g, 3.13 mmol), Pd₂(dba)₃ (120 mg, 0.13 mmol), and P(o-tol)₃ (318 mg, 1.04 mmol) were dissolved in anhydrous toluene (30 mL) and stirred at 110° C. overnight under N₂ atmosphere. After being cooled to room temperature, the reaction mixture was extracted with hexane, washed with aqueous KF solution, water and brine. After concentration under reduced pressure, the crude product was purified by column chromatography (stationary phase: silica gel; eluent: n-hexane/dichloromethane=1/1) to get the product as a light yellow oil (1.19 g, 82%). ¹H NMR (400 MHz, CDCl₃) δ 7.42 (d, J=8.7 Hz, 2H), 7.14-7.05 (m, 4H), 7.02 (s, 1H), 6.98-6.91 (m, 2H), 6.90-6.82 (m, 4H), 6.77 (s, 1H), 3.83 (s, 6H), 2.55 (d, J=6.8 Hz, 2H), 1.70-1.63 (m, 1H), 1.38-1.25 (m, 16H), 0.91 (t, J=6.7 Hz, 6H). ¹³C NMR (101 MHz, CDCl₃) δ 155.88, 148.00, 143.84, 142.89, 140.84, 127.11, 126.49, 126.33, 123.69, 120.82, 119.13, 114.71, 55.50, 38.87, 35.18, 33.39, 33.35, 31.94, 30.04, 29.72, 29.66, 29.37, 26.64, 22.71, 14.14.

### Synthesis of 4-(4-(2-butyloctyl)-5-(tributylstannyl)thiophen-2-yl)-N,N-bis(4-methoxyphenyl)aniline (Compound 4)

To a solution of compound 3 (1.00 g, 1.80 mmol) in anhydrous THE (20 mL) was added n-BuLi (0.86 mL, 2.5 M in hexane) at −78° C. under N₂ atmosphere. The mixture was stirred at the same temperature for one hour, followed by the addition of tributyltin chloride (0.76 g, 2.34 mmol). The reaction was allowed to slowly warm up to room temperature and stirred overnight. The reaction mixture was quenched by aqueous KF solution, washed with water and brine, and dried over Na₂SO₄. The crude product was obtained by concentration under reduced pressure and used without further purification.

### Synthesis of 2-(4-(2,2-bis(4-methoxyphenyl)-1-phenylvinyl)phenyl)-4-(2-butyloctyl)thiophene (Compound 5)

A mixture of 4,4′-(2-(4-bromophenyl)-2-phenylethene-1,1-diyl)bis(methoxybenzene) (1.00 g, 2.13 mmol), compound 2 (1.38 g, 2.55 mmol), Pd₂(dba)₃ (97 mg, 0.11 mmol), and P(o-tol)₃ (259 mg, 0.85 mmol) were dissolved in anhydrous toluene (30 mL) and stirred at 110° C. overnight under N₂ atmosphere. After being cooled to room temperature, the reaction mixture was extracted with hexane, washed with aqueous KF solution, water and brine. After concentration under reduced pressure, the crude product was purified by column chromatography (stationary phase: silica gel; eluent: n-hexane/dichloromethane=1/1) to get the product as a light yellow oil (1.06 g, 77%). ¹H NMR (400 MHz, CDCl₃) δ 7.36 (d, J=8.4 Hz, 2H), 7.15-7.10 (m, 3H), 7.10-7.04 (m, 3H), 7.04-6.99 (m, 4H), 6.99-6.93 (m, 2H), 6.81 (s, 1H), 6.71-6.64 (m, 4H), 3.77 (d, J=3.9 Hz, 6H), 2.54 (d, J=6.8 Hz, 2H), 1.68-1.60 (m, 1H), 1.36-1.24 (m, 16H), 0.94-0.87 (m, 6H). ¹³C NMR (101 MHz, CDCl₃) δ 158.17, 158.08, 144.18, 143.62, 143.40, 142.97, 140.27, 138.70, 136.36, 136.33, 132.61, 132.22, 131.84, 131.46, 127.73, 126.16, 124.77, 124.68, 120.11, 113.14, 112.99, 55.09, 38.84, 35.11, 33.31, 33.01, 31.92, 29.70, 28.87, 26.59, 23.07, 22.69, 14.17, 14.13.

### Synthesis of 4-(4-(2-butyloctyl)-5-(tributylstannyl)thiophen-2-yl)-N,N-bis(4-methoxyphenyl)aniline (Compound 6)

To a solution of compound 5 (1.00 g, 1.56 mmol) in anhydrous THE (20 mL) was added n-BuLi (0.75 mL, 2.5 M in hexane) at −78° C. under N₂ atmosphere. The mixture was stirred at the same temperature for one hour, followed by the addition of tributyltin chloride (0.66 g, 2.02 mmol). The reaction was allowed to slowly warm up to room temperature and stirred overnight. The reaction mixture was quenched by aqueous KF solution, washed with water and brine, and dried over Na₂SO₄. The crude product was obtained by concentration under reduced pressure and used without further purification.

### Synthesis of TPA-TBT

A mixture of 4,8-dibromo-6-(2-ethylhexyl)[1,2,5]thiadiazolo[3,4-f]benzotriazole (100 mg, 0.22 mmol), compound 4 (0.76 g, 0.90 mmol), Pd₂(dba)₃ (10 mg, 0.011 mmol), and P(o-tol)₃ (27 mg, 0.090 mmol) were dissolved in anhydrous toluene (10 mL) and stirred at 110° C. overnight under N₂ atmosphere. After being cooled to room temperature, the reaction mixture was extracted with hexane, washed with aqueous KF solution, water and brine. After concentration under reduced pressure, the crude product was purified by column chromatography (stationary phase: silica gel; eluent: n-hexane/dichloromethane=1/2) to get the product as a dark blue solid (204 mg, 65%). ¹H NMR (400 MHz, CDCl₃) δ 7.52 (d, J=8.7 Hz, 4H), 7.25 (s, 2H), 7.17-7.09 (m, 8H), 7.03-6.94 (m, 4H), 6.92-6.84 (m, 8H), 4.81 (d, J=7.2 Hz, 2H), 3.84 (s, 12H), 2.67 (d, J=7.0 Hz, 4H), 2.47-2.38 (m, 1H), 1.55-1.31 (m, 12H), 1.23-1.12 (m, 4H), 1.10-0.80 (m, 38H), 0.67 (td, J=7.0, 2.5 Hz, 6H). ¹³C NMR (101 MHz, CDCl₃) δ 155.98, 151.96, 148.28, 146.16, 144.93, 143.73, 140.73, 128.34, 126.69, 126.53, 124.92, 120.44, 114.74, 113.87, 61.50, 55.51, 40.66, 39.10, 34.76, 33.29, 32.92, 31.81, 30.52, 29.55, 28.59, 28.46, 26.41, 23.81, 22.97, 22.84, 22.65, 14.09, 14.06, 14.04, 10.42. MS (ESI) m/z calcd. for C₈₆H₁₀₅N₇O₄S₃⁺: 1395.7390. Found: 1395.7391.

### Synthesis of TPE-TBT

A mixture of 4,8-dibromo-6-(2-ethylhexyl)[1,2,5]thiadiazolo[3,4-f]benzotriazole (100 mg, 0.22 mmol), compound 6 (0.79 g, 0.85 mmol), Pd₂(dba)₃ (10 mg, 0.011 mmol), and P(o-tol)₃ (26 mg, 0.085 mmol) were dissolved in anhydrous toluene (10 mL) and stirred at 110° C. overnight under N₂ atmosphere. After being cooled to room temperature, the reaction mixture was extracted with hexane, washed with aqueous KF solution, water and brine. After concentration under reduced pressure, the crude product was purified by column chromatography (stationary phase: silica gel; eluent: n-hexane/dichloromethane=1/2) to get the product as a dark blue solid (215 mg, 61%). ¹H NMR (400 MHz, CDCl₃) δ 7.47 (d, J=8.4 Hz, 4H), 7.35 (s, 2H), 7.18-6.97 (m, 22H), 6.70 (dd, J=15.4, 8.8 Hz, 8H), 4.80 (d, J=7.2 Hz, 2H), 3.79 (d, J=13.0 Hz, 12H), 2.66 (d, J=7.0 Hz, 4H), 2.45-2.36 (m, 1H), 1.54-1.31 (m, 12H), 1.23-1.14 (m, 4H), 1.10-0.81 (m, 38H), 0.68 (td, J=7.0, 2.5 Hz, 6H). ¹³C NMR (101 MHz, CDCl₃) δ 158.24, 158.13, 151.97, 145.77, 144.94, 144.15, 143.83, 143.79, 140.44, 138.76, 136.35, 136.31, 132.66, 132.01, 131.85, 131.50, 129.17, 127.77, 126.21, 125.82, 124.96, 113.90, 113.18, 113.02, 61.68, 55.10, 40.65, 39.08, 34.70, 33.25, 32.89, 31.81, 30.53, 29.55, 28.55, 28.45, 26.37, 23.82, 22.97, 22.84, 22.66, 14.10, 14.06, 14.04, 10.43. MS (ESI) m/z calcd. for C₁₀₂H₁₁₅N₅O₄S₃Na⁺: 1593.8037. Found: 1593.8024.

### Example 2—Photophysical, Photodynamic, and Photothermal Properties

First, the absorption and emission spectra were recorded. From FIG. 3a, the maximum absorptions of TPE-TBT and TPA-TBT are located at 582 and 620 nm, respectively, which display a redshift because of the enhancement of D-A strength from TPE-TBT to TPA-TBT. In addition, the emission spectra of the two molecules peaked at 750 and 830 nm, respectively (FIG. 3b). TPA-TBT shows an enormous Stokes shift of 210 nm, which benefits real applications. The aggregation behaviors of the compounds were then investigated. After adding poor solvent water to the THE solution, the fluorescence intensities of the two luminogens are first reduced at low water fractions (0-50% of water) resulting from the twisted intramolecular charge transfer (TICT) effect. With the water fraction increasing, the fluorescence emission intensities of both compounds are gradually enhanced, ascribed to the typical AIE effect. Significantly, TPA-TBT displays a more obvious AIE property with a 3-fold intensity enhancement (FIG. 3c).

For better use in cancer theranostic, TPA-TBT and TPE-TBT were fabricated into nanoparticles (NPs) through the nanoprecipitation method using DSPE-PEG₂₀₀₀ as an encapsulation matrix. The hydrodynamic diameters of the two NPs were confirmed by dynamic light scattering (DLS). The result shows the TPA-TBT and TPE-TBT NPs are well dispersed in water with diameters of around 37 nm and 45 nm, respectively (FIG. 4 and FIG. 5). The UV/Vis absorption and fluorescence emission spectra of the two kinds of NPs were also measured, and the results are similar to that in the THF solutions. The fluorescence quantum yield (QY) of the two compounds in the solid state, NPs, THF, and other solvents, were also measured (FIG. 3d). The result shows that the TPA-TBT NPs have a high absolute QY of 7%, while TPE-TBT NPs have an even higher QY of 28%. The QY of TPA-TBT in the solid state is 13%, which is higher than that in NPs and THE solution (5%), while TPE-TBT has remarkable QY in the solution state, indicating TPE-TBT shows a more significant TICT effect and active radioactive decay. ROS production efficiency and PCE were investigated to further explore the phototherapy potential of these AIEgens. We utilized a common ROS indicator Dichlorofluorescin Diace (H₂DCF-DA), whose fluorescence can be sensitively activated by ROS, to indicate the overall ROS generation. From FIG. 3e, the fluorescence intensity of H₂DCF-DA intensified rapidly in the presence of TPA-TBT NPs. Finally, it reached around 210-fold more robust than the initial intensity after 5 min upon white light irradiation, displaying a much higher ROS production efficiency than TPE-TBT NPs. In addition, TPA-TBT NPs exhibit better photothermal performance. As shown in FIG. 3f, the temperature of TPA-TBT NPs reached around 57° C. upon the irradiation of 660 nm laser in the 350s, with a PCE of 44%. While the PCE of TPE-TBT NPs is only 30%. Considering all the aspects, TPA-TBT NPs show longer fluorescence emission wavelength, higher ROS generation efficiency, and better photothermal efficiency. Therefore, TPA-TBT NPs are more promising for applications in the field of phototheranostic for synergistic cancer therapy.

### Example 3—Cellular Imaging

The cytotoxicity of the TPA-TBT NPs in dark was studied by CCK-8 assay to explore the biocompatibility of synthetic probe. The results shown in FIG. 6a demonstrated that TPA-TBT NPs have no obvious dark toxicity toward normal cells (human embryonic kidney cells 293T) and tumor cells (human cervical carcinoma cells Hela and mouse breast cancer cells 4T1) even at a very high concentration (100 ug/mL). However, with the extra treatment of weak white light irradiation, TPA-TBT NPs exhibited evident phototoxicity toward the cells (FIG. 6b). Even at a low concentration of the NPs, the cell viability still significantly reduced. These observations indicate that the TPA-TBT NPs possess excellent biocompatibility and efficient light-killing effect. Flow cytometry and confocal imaging further confirmed the light killing efficiency to cancer cells, suggesting that the PTT and PDT combined outcome of TPA-TBT NPs was significantly effective with a total killing rate of 67.3%. The 4T1 cells from the other groups, including cells without treatment, treated with light irradiation, and treated with NPs, showed negligible apoptotic or necrosis portions in the whole cell population (FIG. 6c).

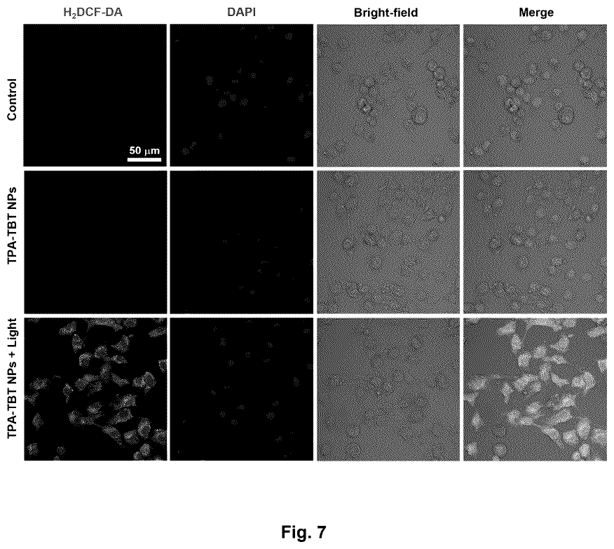

Furthermore, the intracellular ROS generation was investigated by H₂DCF-DA in 4T1 cells upon irradiation. As shown in FIG. 7, 4T1 cells treated with TPA-TBT NPs and H₂DCF-DA displayed strong green fluorescence upon weak white light irradiation. In contrast, no fluorescence was detected in PBS buffer (control) nor single TPA-TBT NPs treatment without laser. These results demonstrate that TPA-TBT NPs can efficiently generate ROS to support excellent phototoxicity against cancer cells.

### Example 4—In Vivo Imaging-Guided Cancer Therapy

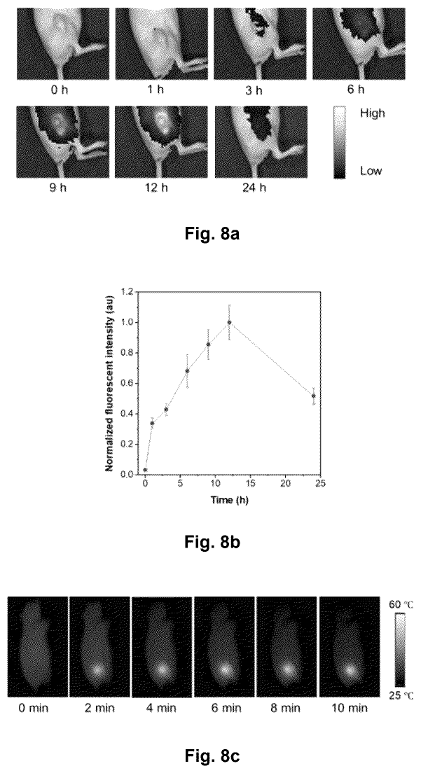

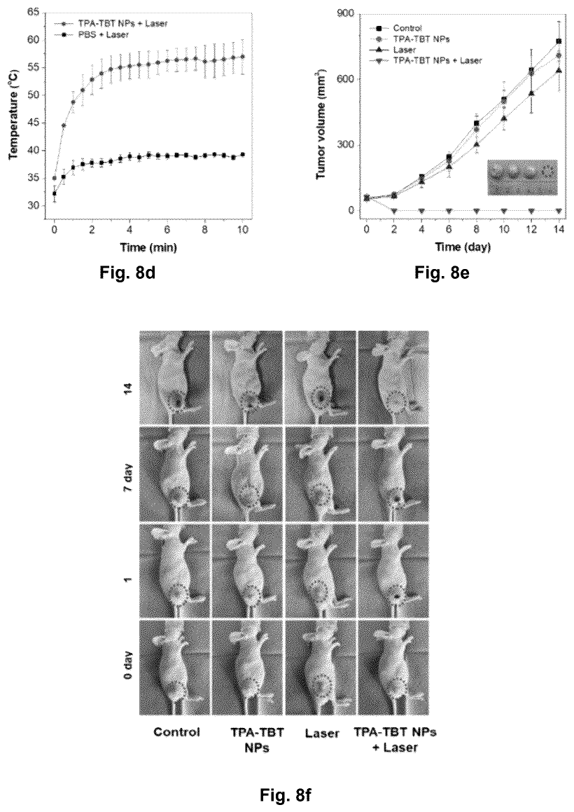

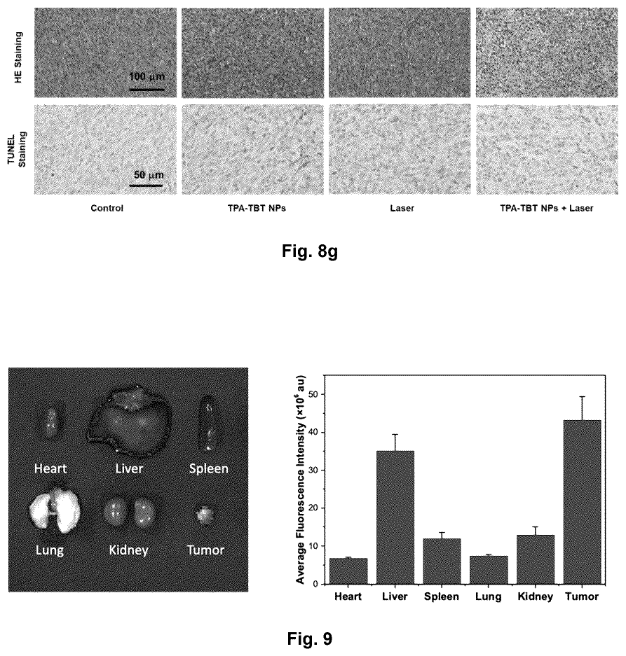

To explore the in vivo FLI and tumor accumulation capability of TPA-TBT NPs, the NPs were first injected into the 4T1 tumor-bearing mice intravenously. And the fluorescence images of the mice were obtained at different time intervals after injection. As displayed in FIGS. 8a and 8b, the fluorescence signal gradually intensified with the extension of time. It reached the strongest at 12 h, indicating the TPA-TBT NPs could accumulate at the tumor site and display excellent performance in FLI. To further investigate the biological distribution of TPA-TBT NPs, the main organs (heart, liver, spleen, lung, and kidney) and tumors of mice were collected after 12 h injection of the NPs. The results showed that TPA-TBT NPs mainly accumulated in the tumor and liver. The tumor tissue exhibited the strongest fluorescence signal, indicating the tumor accumulation and hepatic metabolism effect of TPA-TBT NPs (FIG. 9).

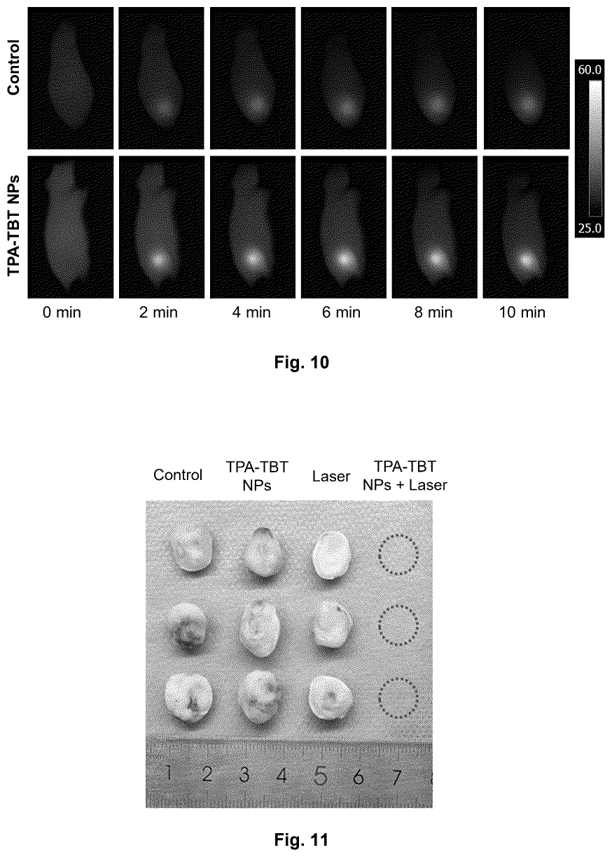

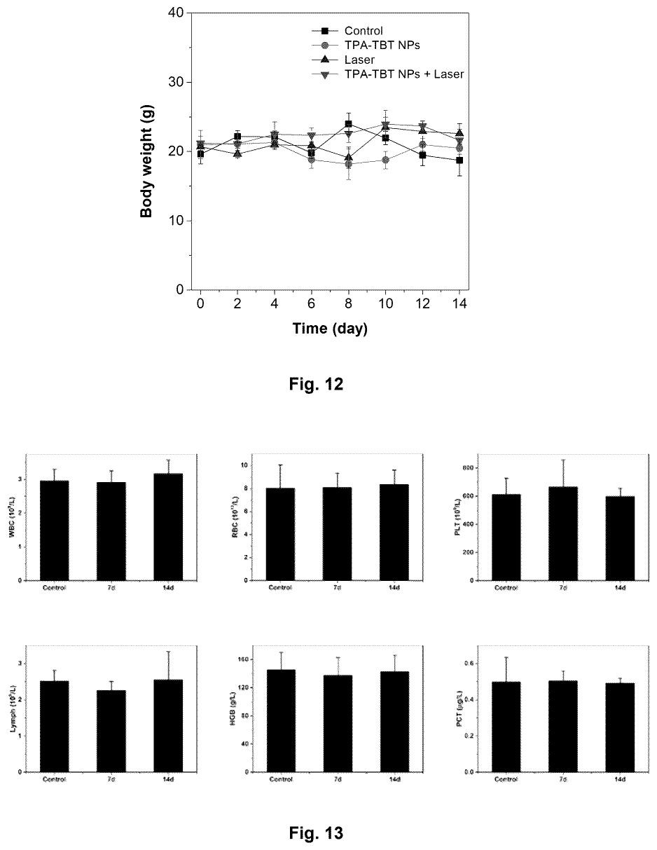

Then we explored the in vivo phototheranostic performance and antitumor capacity of the TPA-TBT NPs in the 4T1 tumor-bearing mice. First, the tumor temperature of the mice treated with TPA-TBT NPs rose rapidly from 35° C. to above 50° C. in 2 min and reached around 57° C. within 10 min upon laser irradiation. In comparison, the tumor temperature of the control group (only treated with PBS buffer) showed no significant changes (FIG. 8c, 8d and FIG. 10). To investigate the continuous therapeutic efficiency of the TPA-TBT NPs, 4T1 tumor-bearing nude mice were randomly divided into four groups for different treatments. As shown in FIG. 8e and FIG. 11, the tumors were photographed before (0 days) and after (1, 7, and 14 days) the injection of TPA-TBT NPs. Also, the tumor sizes were monitored every two days to assess the therapeutic efficiency of each group. The results exhibited that tumors were completely eradicated in the group treated with TPA-TBT NPs and irradiated with 660 nm laser (0.5 W cm⁻²), while the tumors constantly grew without noticeable inhibition in the other three groups. In addition, the bodyweight of the mice in all groups showed no significant loss in 14 days, indicating the great biosafety of our therapeutic approach with TPA-TBT NPs (FIG. 12).

Furthermore, to investigate the tumor-killing mechanism in the phototherapeutic process, hematoxylin and eosin (H&E) staining and the terminal deoxynucleotidyl transferase dUTP nick end labeling (TUNEL) staining were conducted (FIG. 8f). Compared with the three control groups, the tumor cell density in the group treated with TPA-TBT NPs and laser decreased sharply. Besides, the TUNEL staining result further verified that TPA-TBT NPs plus laser irradiation could produce ROS and induce tumor cell apoptosis. These results indicate that TPA-TBT NPs induce tumor obliteration through cell necrosis and apoptosis contributed by the simultaneous PTT and PDT effects under light irradiation.

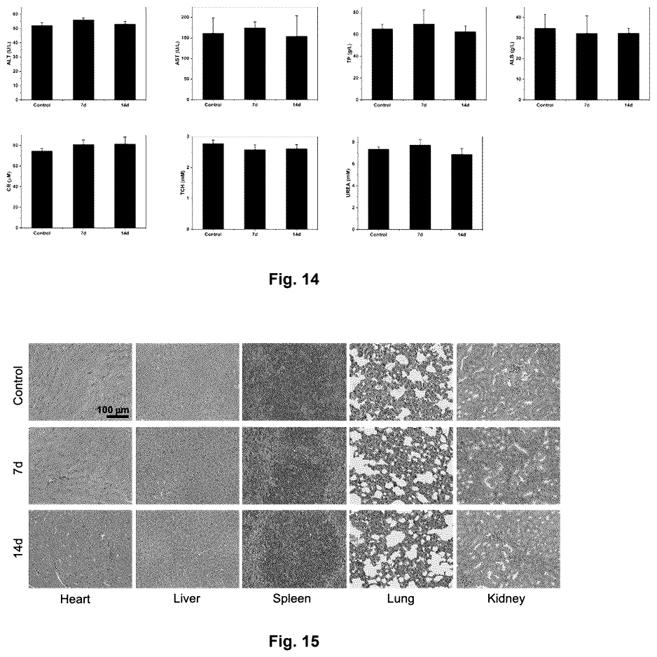

Furthermore, the in vivo safety of NPs was systematically evaluated. The mice were injected with TPA-TBT NPs (10 mg/kg, 200 μL). The blood routine and biochemical analyses were conducted 7 and 14 days after injection and compared with the control group, which was treated with PBS (FIGS. 13 and 14). From the analysis, all the parameters are at normal levels compared with the control groups. The main organs were collected at different time points for H&E staining, and no abnormality could be found after TPA-TBT NPs treatments (FIG. 15). These results demonstrate that TPA-TBT NPs exhibit excellent in vivo biocompatibility, which could be widely used as a powerful fluorescent imaging-guided PTT and PDT agent for cancer phototheranostics.

The above embodiments are only used to illustrate the principles of the present invention, and they should not be construed as to limit the present invention in any way. The above embodiments can be modified by those with ordinary skill in the art without departing from the scope of the present invention as defined in the following appended claims.

What is claimed is:

1. A fluorescent compound exhibiting aggregation-induced emission properties, the fluorescent compound having a backbone structural formula selected from the group consisting of: 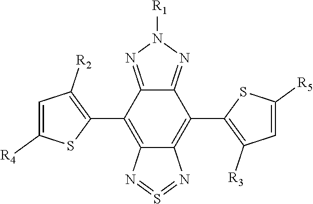
wherein each R₁, R₂, and R₃ is independently selected from the group consisting of straight-chain, branched, cyclic alkyl, alkyl phenyl, alkyl thienyl and other alkyl aryl with 2-40 C atoms, wherein one or more non-adjacent C atoms are optionally replaced by —O—, —S—, —C(O)—, —C(O—)—O—, —O—C(O)—, —O—C(O)—O—, —CR5=CR6-, or —C≡C—, and wherein one or more H atoms are optionally replaced by F, Cl, Br, I, or CN or denote aryl, heteroaryl, aryloxy, heteroaryloxy, arylcarbonyl, heteroarylcarbonyl, arylcarbonyloxy, heteroarylcarbonyloxy, aryloxycarbonyl, or heteroaryloxycarbonyl having 4 to 30 ring atoms unsubstituted or substituted by one or more non-aromatic groups;
wherein each R₄ and R₅ is independently selected from the group consisting of:

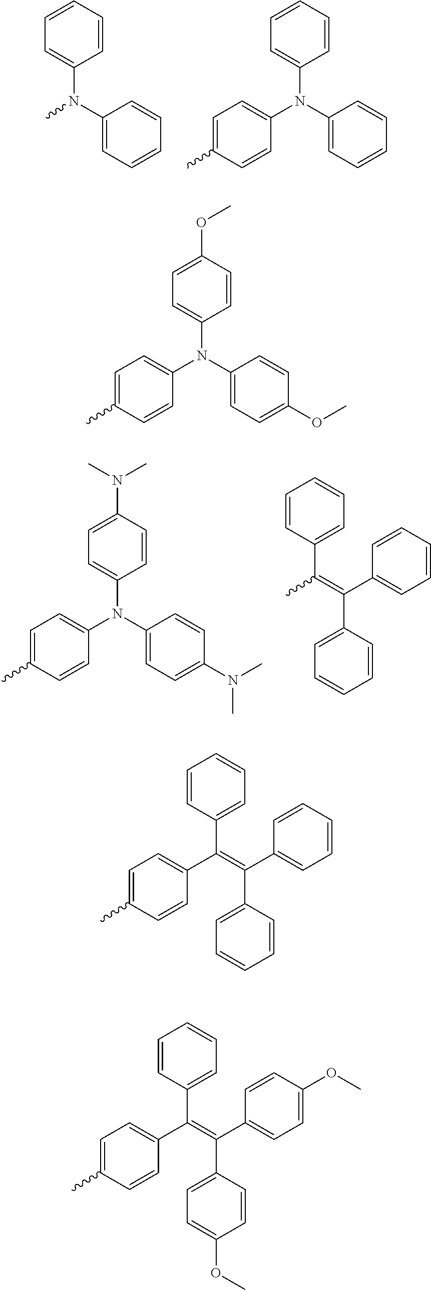

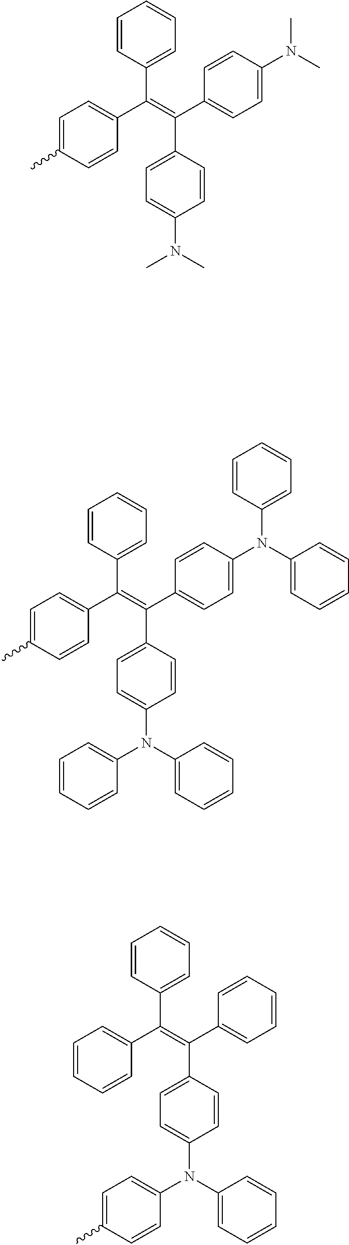

2. The fluorescent compound of claim 1, wherein R₁, R₂, and R₃ are branched alkyl with 2-40 C atoms.

3. The fluorescent compound of claim 1, wherein the fluorescent compound is selected from the group consisting of:

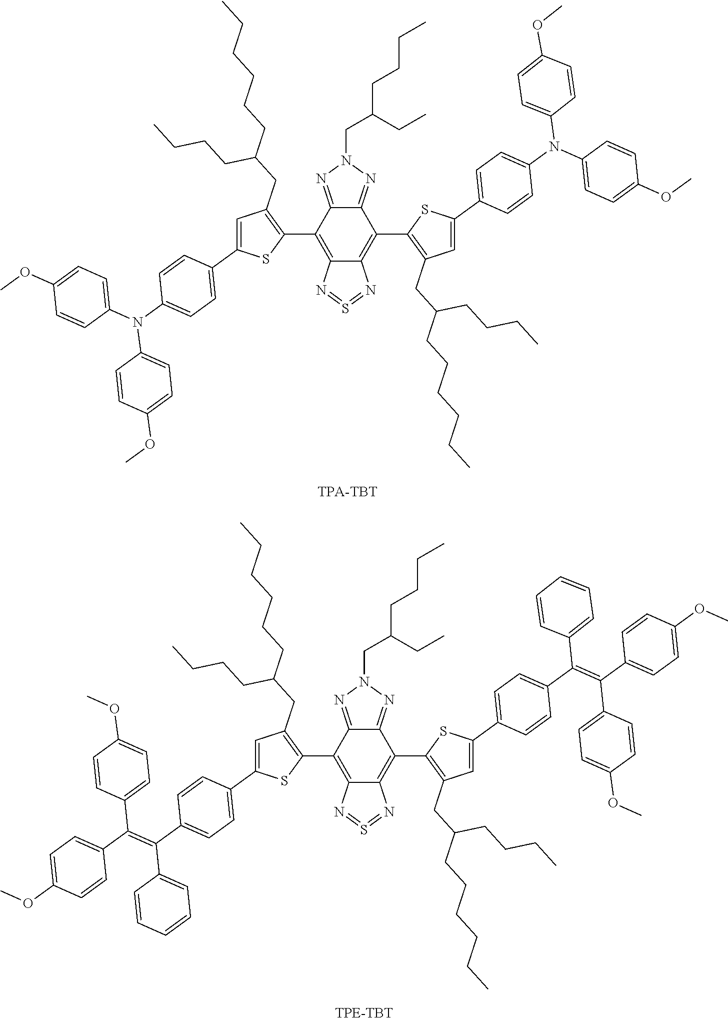

4. The fluorescent compound of claim 1, wherein the fluorescent compound exhibits near infrared emission.

5. The fluorescent compound of claim 1, wherein the fluorescent compound is in nanoparticle form.

6. A theranostic agent comprising the fluorescent compound of claim 1 in nanoparticle form and a polymer matrix encapsulating the fluorescent compound.

7. A method of killing cancer cells, comprising:
contacting a target cancer cell with the theranostic agent according to claim 6;
imaging the target cancer cell while the theranostic agent contacts the target cancer cell using an imaging method selected from the group consisting of fluorescence microscopy, bioluminescence imaging, and confocal laser scanning microscopy; and
subjecting the target cancer cell to light irradiation while the theranostic agent is contacting the target cancer cell to kill the target cancer cell.

8. The method of claim 7, wherein subjecting the target cancer cell to light irradiation while the theranostic agent is contacting the target cancer cell absorbs light and generates reactive oxygen species to kill the target cancer cell.

9. The method of claim 7, wherein subjecting the target cancer cell to light irradiation while the theranostic agent is contacting the target cancer cell converts light to heat to kill the target cancer cell.

10. The method of claim 7, wherein the target cancer cell is in live mammals.

11. The method of claim 7, wherein the light irradiation is white light irradiation.

12. The method of claim 7, wherein the light irradiation is red light irradiation.

13. The method of claim 7, wherein the light irradiation is laser irradiation.

14. A method of stopping or inhibiting tumor growth in a mammal, comprising:
administering the theranostic agent of claim 6 to the mammal;
contacting a tumor site with the theranostic agent;
locating the tumor site using an imaging method after the tumor site is contacted with the theranostic agent; and
subjecting the tumor site to light irradiation while the theranostic agent is present at the tumor site to stop or inhibit the growth of the tumor.

15. The method of claim 14, wherein subjecting the tumor site to light irradiation while the theranostic agent is present at the tumor site absorbs light and generates reactive oxygen species to stop or inhibit the growth of the tumor.

16. The method of claim 14, wherein subjecting the tumor site to light irradiation while the theranostic agent is present at the tumor site converts light to heat to stop or inhibit the growth of the tumor.

17. The method of claim 14, wherein the theranostic agent is administered by injection.

18. The method of claim 14, wherein the imaging method comprises at least one of fluorescence microscopy, bioluminescence imaging, and confocal laser scanning microscopy.

19. The method of claim 14, wherein the light irradiation is white light irradiation.

20. The method of claim 14, wherein the light irradiation is red light irradiation.

21. The method of claim 14, wherein the light irradiation is laser irradiation.
